(19) United States
(12) Patent Application Publication
(10) Pub. No.: US 2022/0134007 A1
(43) Pub. Date: May 5, 2022

(54) DEVICE, METHOD AND SYSTEM FOR ANALGESIC DELIVERY INTO GINGIVA

(71) Applicant: Mateo Vidales, Lakeland, FL (US)

(72) Inventor: Mateo Vidales, Lakeland, FL (US)

(21) Appl. No.: 17/451,865

(22) Filed: Oct. 22, 2021

Related U.S. Application Data
(60) Provisional application No. 63/108,840, filed on Nov. 2, 2020.

(51) Int. Cl. A61M 5/28; A61M 19/00; A61M 5/32

(52) U.S. Cl. CPC A61M 5/282; A61M 2037/0023; A61M 5/3298; A61M 19/00

(57) ABSTRACT

A device, a method, and a system for non-intrusive percutaneous delivery of analgesia into gingiva is disclosed. The disclosure includes a frame supporting a pressable reservoir filled with liquid anesthetic and has a plurality of hollow needles terminating at or adjacent a base portion. The method may have one or more two modes. This dual modality includes a first mode, wherein a topical anesthetic disposed along or adjacent the base portion may be applied to the gingiva. During a second mode, the pressable reservoir is manually compressed, whereby the needles are urged toward the patient, entering the gingiva and delivering the anesthetic. These two modes provide for an anesthetic process at the beginning of dental procedures in preparation for the final larger dose using a hypodermic needle.

### CROSS-REFERENCE TO RELATED APPLICATION

This application claims the benefit of priority of U.S. provisional application No. 63/108,840, filed Nov. 2, 2020, the contents of which are herein incorporated by reference.

### BACKGROUND OF THE INVENTION

The present invention relates to the dental arts and more particularly, to a device, a method, and a system for delivering anesthetic to gingiva. The present invention embodies a pressable reservoir that delivers initial dental anesthesia prior to a hypodermic needle injection of anesthetic, thereby reducing the pain of the latter.

It is believed that the first local anesthesia was developed by the Peruvians. They had long known that chewing on coca leaves caused a numbing of the mucous membrane in the mouth. It was not until the latter half of the 19th century that research into this effect was conducted in Europe. This research lead to the first eye operation under local anesthesia by the eye surgeon, Koller in Vienna in 1884. The anesthesia was achieved using cocaine. Following this first successful operation cocaine was increasingly administered as a local anesthetic. In fact, cocaine became commonplace in Europe as a local anesthetic. Before long, the disadvantages of cocaine became self-evident. Its short-lasting effect and addiction were problematic. However, the local anesthesia phenomenon had become well known and greatly appreciated. It was now possible to carry out an operation without causing the patient unnecessary suffering, the doctor was better able to concentrate on the procedure in hand and the affliction could be treated much sooner because the threshold for both doctor and patient to start treatment had become much lower.

Accordingly, the need arose to find alternatives for cocaine. An alternative appeared in 1905 in the form of Procaine. It was marketed under the name Novocain and remained the most important local anesthetic until the 1940's. Novocain was and is (it is still being sold) an ester-type anesthetic. It is a cocaine derivative, meaning that it is chemically related to cocaine, with similar characteristics, but without the toxicity, with a longer-lasting effect and without the problems of addiction.

Another analgesic called lidocaine was first developed in 1943 and marketed in 1947 under the name, Xylocaine. It was the first amide-type local anesthetic, meaning that it was broken down in the liver rather than in the bloodstream. Lidocaine is generally well tolerated by patients, is mildly toxic, is long-lasting effect and is non-addictive. The only disadvantage of lidocaine is that it is slow in taking effect. In 1976, articaine was developed and marketed under the trade name Ultracain by Hoechst A.G. and shortly thereafter, under the name Septanest by the French pharmaceutical company, Septodont S.A. This last product has the same characteristics as Ultracain. In 1983 Ultracain was approved for the Dutch market. In this time, it is estimated that Ultracain and Septanest have jointly attained a 40-45% share of the market. Since then, Scandonest has also been approved as a local anesthetic for dental use. The active component in Scandonest is scandicaine. Scandicaine is a substance originally used in regional anesthesia (epidural anesthesia) and developed into a local anesthetic, which as far as we know has no negative side effects. This substance does not use a vasoconstrictor and as such does not need any preservative. While these analgesics does a good job at making a patient feel comfortable during a dental procedure the administration of them can be discomforting if not painful for some, thereby tending to increase the anxiety of some patients when ideally the patient is relaxed and calm.

These analgesics are currently administered by way of an elongated hypodermic needle into the gingiva. With little doubt, the length of the hypodermic needle itself plays a role in making patients anxious prior to the administration of the analgesic as well as uncomfortable during it. What is needed is a method, device, and system to minimize discomfort to the gingiva during the insertion of a hypodermic needle therein through a prophylactic delivery of analgesia.

Current solutions have yet to provide a painless solution to injecting anesthetics, and so at best involve topical analgesics prior to injecting anesthesia via the hypodermal needle straight into gingiva. There are limits to the pain-reducing effects of topical analgesics, and so preferably an intermediate percutaneous application of anesthesia, prior to the hypodermic needle application. The previous methods, rather, use the same large needle but just deliver a smaller amount of anesthesia. As mentioned above, the combination of the size and the pain of the hypodermal needle can be discomforting and anxiety producing. Furthermore, applications of hypodermically delivered analgesics can provide discomfort to the patient due to the stretching of the gingiva caused by the injection of fluid. In sum, the typical methods of anesthesia have not changed in decades and are a main reason people fear going to the dentist.

Accordingly, there is a need for a device, a method, and a system for percutaneously delivering anesthetic to gingiva before a hypodermic needle injection in a minimally invasive manner. The present invention embodies a pressable reservoir that delivers initial dental anesthesia via, in some embodiments, through two modes by way of one device, thereby reducing pain for patient at site of hypodermic needle, as well as providing the percutaneously delivery in a physically smaller form than a convention hypodermic needle, and thus less to reduce anxiety in the patient. And in some applications, for instance for children, the present invention may obviate the need for the hypodermic needle to administer sufficient anesthesia for a dental patient.

The present invention is dimensioned and adapted to percutaneously deliver an initial anesthesia so that a subsequent hypodermic-needle administration, if still needed, can then be inserted less-painfully to provide profound anesthesia. And in some application, the amount contained in the pressable reservoir may be enough for procedures that do not require profound anesthesia, or for a child.

### SUMMARY OF THE INVENTION

In one aspect of the present invention, a device including the following: a pressable reservoir having a plurality of outlets, wherein the pressable reservoir is movable between an undeformed condition and a deformed condition; and a frame having a support portion spaced apart from a base portion, wherein an upper surface of the support portion is connected to a bottom surface of the pressable reservoir, wherein moving from the undeformed condition to the deformed condition reduces a volume of the pressable reservoir and moves the support portion toward the base portion.

In another aspect of the present invention, the device further including the following: a plurality of needles fluidly coupled to the plurality of outlets, wherein the termini of the plurality of needles is adjacent to bottom surface; a layer of topical gelatinous anesthesia disposed along the base portion in such a way that said layer terminates at a contact surface approximately flush with the bottom surface, wherein said termini move from upward of the contact surface to downward thereof upon moving between the undeformed condition to the deformed condition; a cavity along the base portion, wherein the cavity houses said layer, wherein the frame has curved ends interconnecting the base portion and the support portion, wherein the frame comprises two laterally spaced apart rails; an absorptive material adjacent said layer; and a liquid anesthetic housed in the pressable reservoir.

In yet another aspect of the present invention, a method for delivering analgesic into a gingiva, the method including the following: contacting a layer of the above-mentioned device against a surficial surface of the gingiva for between two to six minutes; and thereafter moving the pressable reservoir to the deformed condition.

These and other features, aspects and advantages of the present invention will become better understood with reference to the following drawings, description and claims.

### BRIEF DESCRIPTION OF THE DRAWINGS

FIG. 1 shows an elevation view of the device in use according to an exemplary embodiment, the device shown in an unpressed/initial condition, illustrating an embodiment wherein the terminus of each needle is just upward of a bottom surface 45 which interfaces with the gingiva of the patient.

FIG. 2 shows a top perspective view of the device in use according to an exemplary embodiment.

FIG. 3 shows a bottom perspective view of the device in use according to an exemplary embodiment.

FIG. 4 shows an end elevation view of the device in use according to an exemplary embodiment.

FIG. 5 shows a top perspective view of the device in use according to an exemplary embodiment.

FIG. 6 shows a side elevation view of the device in use according to an exemplary embodiment.

FIG. 7 shows a bottom perspective view of the device in use according to an exemplary embodiment.

FIG. 8 shows a bottom perspective view of the device in use according to an exemplary embodiment.

FIG. 9 shows an elevation view of the device in use according to an exemplary embodiment.

FIG. 10 shows an elevation view of the device according to an exemplary embodiment.

FIG. 11 shows a schematic view of an application area of an exemplary embodiment of the present invention.

FIG. 12 shows a schematic view of an exemplary embodiment of the present invention in use, illustrating the dual-mode operation of the device wherein a topical anesthetic may be applied to the gingiva.

### DETAILED DESCRIPTION OF THE INVENTION

The following detailed description is of the best currently contemplated modes of carrying out exemplary embodiments of the invention out in various ways which will be obvious to those skilled in the art. The description, phraseology, and terminology is not to be taken in a limiting sense but is made merely for the purpose of illustrating the general principles of the invention, since the scope of the invention is best defined by the appended claims.

Broadly, an embodiment of the present invention provides a device, a method, and a system for delivering anesthetic to gingiva for maximizing patient comfort and minimizing patient anxiety before hypodermic needle insertion, or even in lieu of the hypodermic needle administration of anesthetic for dental procedures.

The analgesic delivery device 100 embodied in the present invention has a plurality of micro needles 20 that deliver an anesthetic to the surficial layers 80 of the gingiva. The analgesic delivery device 100 may include of a pressable reservoir 10 that contains a liquid anesthetic 30 such as lidocaine or the like. The pressable reservoir 10 provides at least one outlet 12 fluidly coupled to a proximal end of each needle 20.

The analgesic delivery device 100 may have a frame 40 that provides a support portion 42 and a base portion 44. The frame 40 may include two spaced apart rails 46 sufficiently spaced apart to accommodate each outlet 12. In other embodiments, the support portion 42 may be solid but for the outlets 12. The support portion 42 supports the pressable reservoir 10. The base portion 44 may include two spaced apart rails 46 sufficiently spaced apart to accommodate the distal end of each needle 20 to protrude therebetween. In other embodiments, the base portion 44 may be solid but for apertures dimensioned and adapted so that the associated needle 20 can slide therethrough. The base portion 44 provides a bottom surface 45 which interfaces with the surficial layers 80 of the gingiva (or, in other applications, dermal surface) of the patient. In some embodiments, the terminus of each distal end of each needle 20 may be just upward (or away from) the surficial layer 80 of the patient when not in use (or an unpressed/initial condition) or the bottom surface 45. In other embodiments, when not in the initial condition, the distal ends of the needles 20 protrude to and beyond the interfacing bottom surface 45. In the initial condition, a proximal portion of the micro needles 20 may retract inside the reservoir 10.

The pressable portion may be an upper surface or portion of the reservoir 10, or otherwise be a portion or percentage of a reservoir's surface. In other embodiments of the present invention, the entire reservoir may be pressable. It is understood that for the present invention, the term “pressable” refers to an ability of a material to deform, at least partially, in response to applied pressure to the material, whereby the deformation of the pressable material may result in a change of volume in a region contained, at least partially, by the pressable material (as the pressable material is urged against the support portion 42). The pressable material may be made of an elastic polymers or plasticized material, such as silicone or polyurethane, rubber the like. The reservoir 10 may thus be moveable (or pressed) between an unpressed/initial condition and a pressed/deformed condition.

The deformed condition or action of pressing on the pressable portion of the reservoir 10 causes one or more responses. First, the fluid 30 contained within the reservoir 10 may be urged through outlets 12, wherein the outlets 12 are fluidly coupled the plurality of needles 20. And, second, the distal end of the plurality of needles 10 move downward relative to the bottom surface 45, thereby either protrude beyond the bottom surface for the first time or protruding further than the initial condition (relative to the bottom surface 45). The movement of the termini of the plurality of needles 20 is facilitated by the structure of the frame 40, which is adapted to flex in the pressed condition. Specifically, the force of the applied pressure to the pressable material and/or applied pressure to the upper portion of the frame 40, moves the support portion 42 downward relative to the bottom surface 45 which is essentially against the braced the surficial layers of the patient. The curved ends 48 of the frame 40 as well as the spaced apart rails 46 and void that separates the support portion 42 and the base portion 44 enable this frame flex, which in turn urges the distal ends of the needles 20 in the downward direction relative to the bottom surface 45. In certain embodiments, the frame 40 (and in effect the analgesic delivery device 100) is approximately 1.5″ in length×0.5″ width×0.5 height.

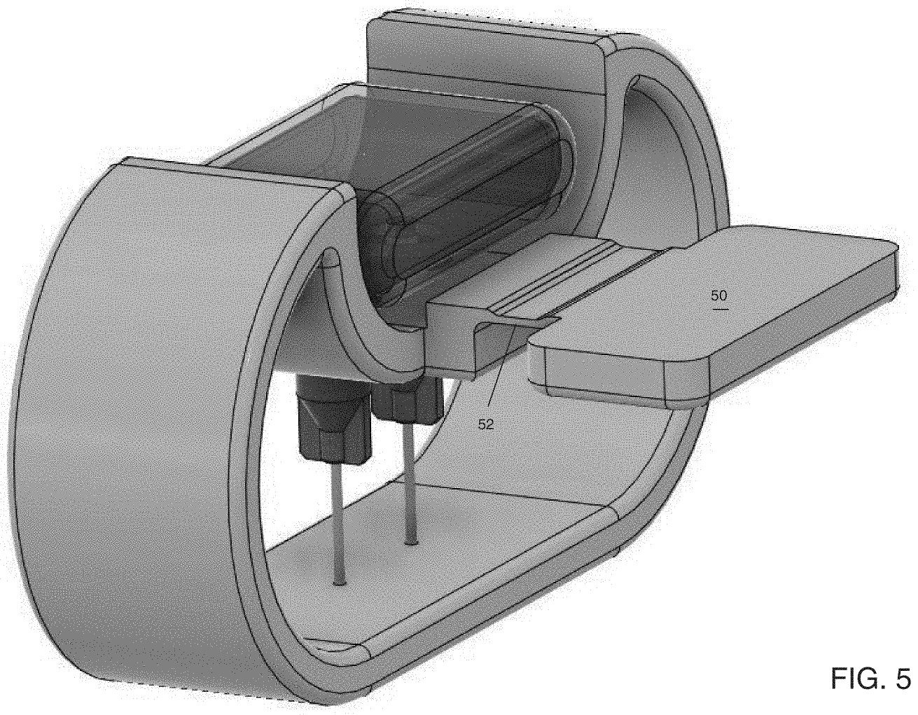

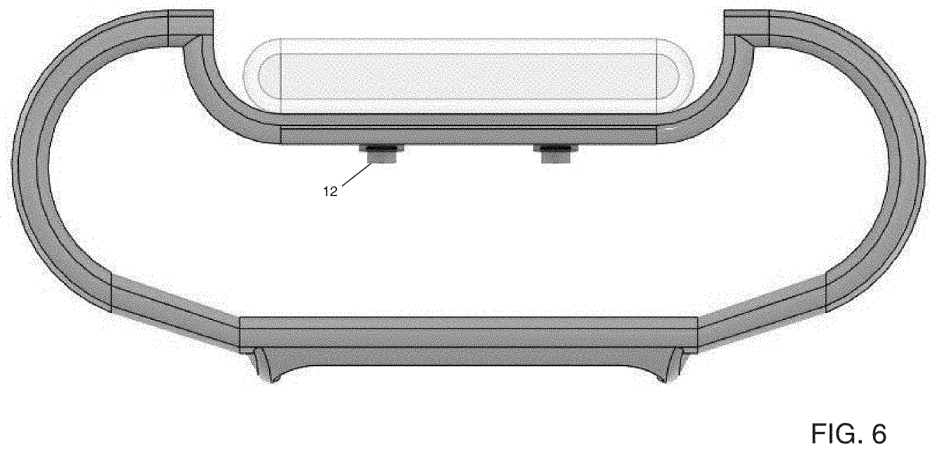

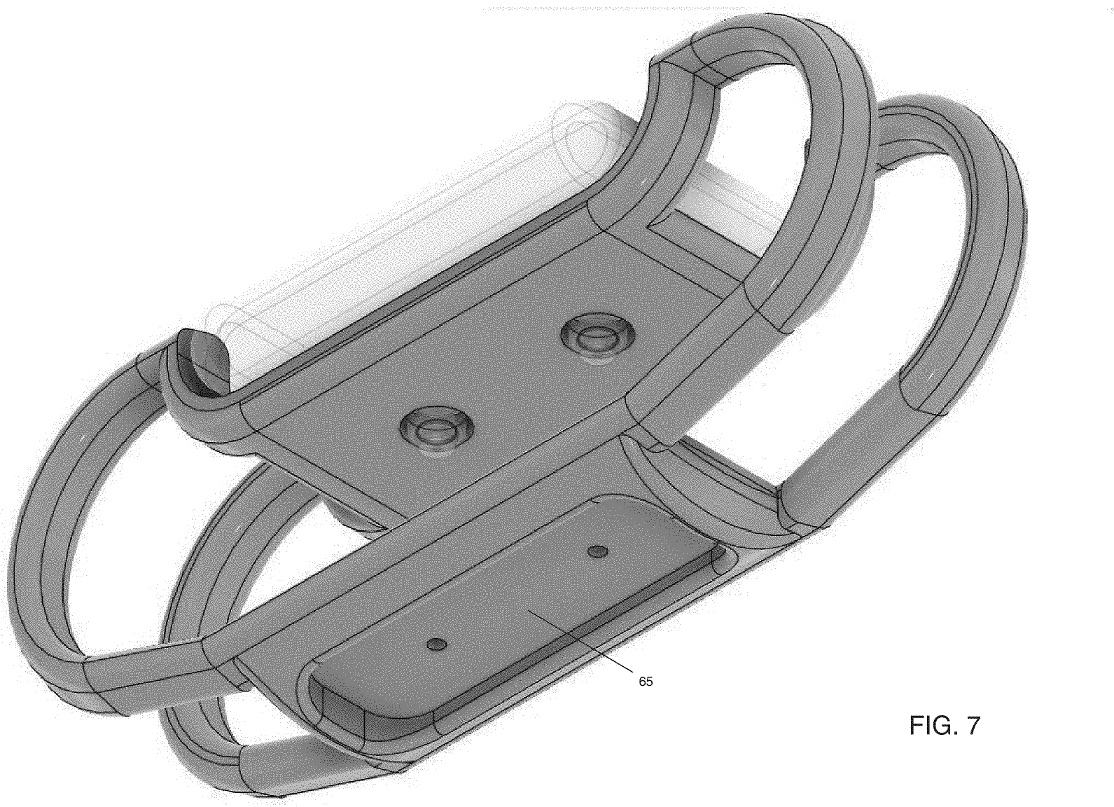

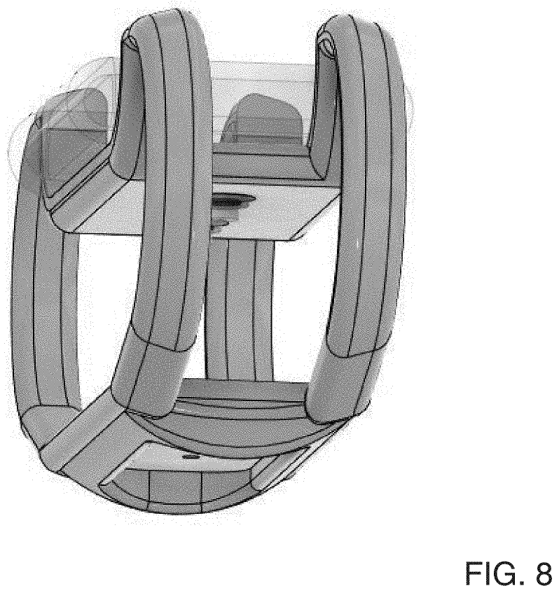

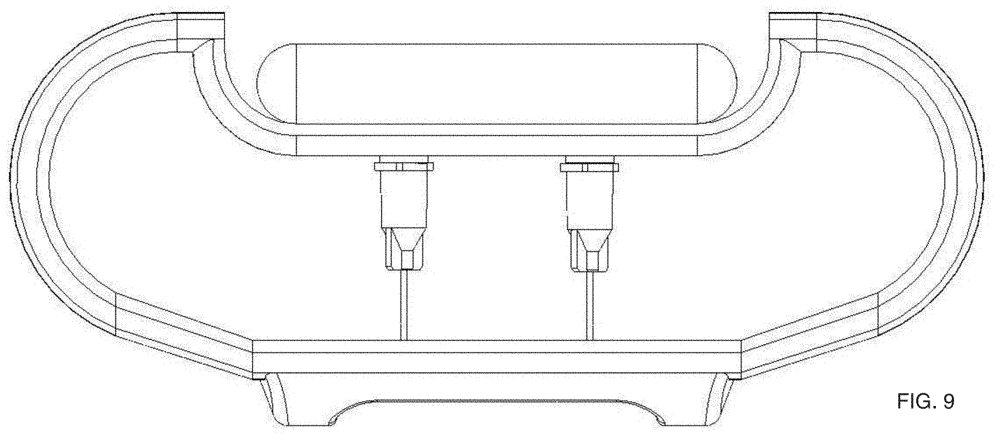

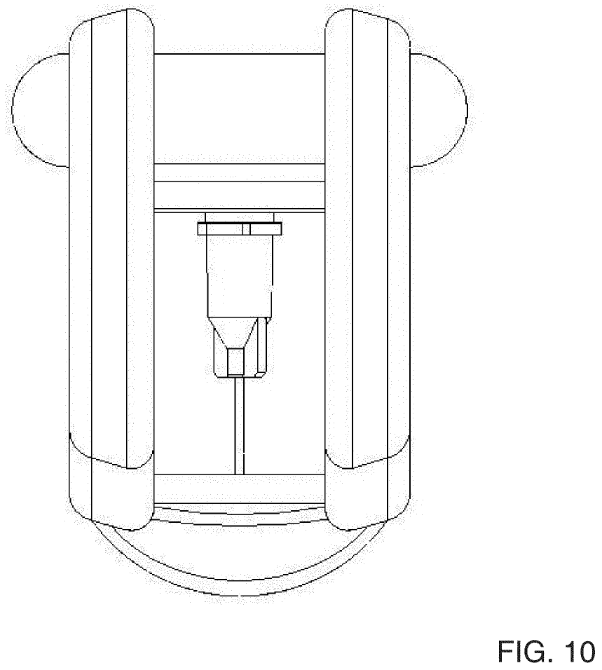

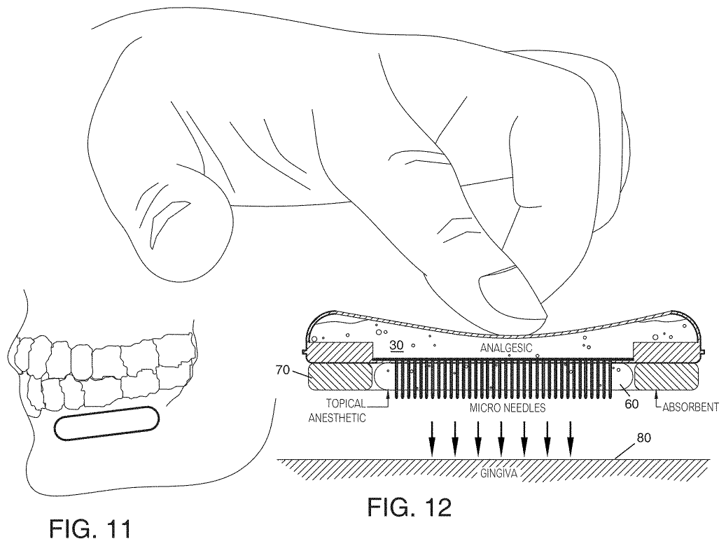

In certain embodiments, a cover 50 may be pivotably connected to the frame 40 in such a way as to move between an exposed position and a guarded position, preventing access to the pressable portion of the reservoir 10, and thus preventing unintentional discharge or delivery of the fluid from the reservoir 10. FIG. 5 illustrates the exposed position whereby the pressable portion of the reservoir is accessible. The pivotable connection 52 of the cover 50 may be by way of a living hinge or other pivotal structures.

In certain embodiments, the analgesic delivery device 100 has two methods of providing anesthesia or anesthetic providers. The first anesthetic provider/mode may include a layer of gelatinous topical anesthesia 60, such as but not limited to a benzocaine mix, a mix of other topical anesthetics, or the like. This gelatinous layer of topical anesthesia 60 may be disposed on the underside of the device 100/frame 40 that rests on the gingiva—i.e., along the bottom surface 45 or adjacent the bottom surface 45. The analgesic delivery device 100 may provide a cavity 65 for the gelatinous topical anesthesia 60, so that a distal surface thereof is approximately flush with the bottom surface 45. Accordingly, as the bottom surface of the gelatinous topical anesthesia 60 engages/contacts the surficial layer 80 of the patient, the topical anesthesia 60 initially takes effect before the device 100 is moved to the pressed/deformed condition, and thus before causing the needles 20 puncture the surficial layer 80—i.e., the second anesthetic provider/mode. In certain embodiments, the plurality of needles 20 protrude through the topical gelatinous layer 60, as illustrated in FIG. 12. The analgesia of the topical gelatinous layer 60 may make it so that the patient cannot feel the needles 20 come through the gingiva delivering the second mode of anesthesia delivery.

In addition to the gelatinous layer of topical there should also be some absorptive material 70 along the base portion 44 to absorb saliva to allow the topical to take effect. The absorptive material 70 may be cotton, absorptive foam or the like.

The device 100 may be disposable after each use. The frame 40 and anesthetic reservoir 10 may be injection molded or additively manufactured, while the custom micro needles 20 would most likely need to be machined. The micro needles 20 may be made of a hollow, rigid material such as but not limited to stainless steel, plastic, and the like.

A method of using this embodiment of the present invention may include the following. The practitioner first engages the bottom layer 45/topical gelatinous layer 60 to the surficial layer 80 of the gingiva, waiting approximately three to five minutes until the topical anesthesia 60 takes action. Thereafter, the practitioner moves the reservoir 10 to the pressed condition via applying pressure through their fingers. This compression forces the needles 20 to break through the bottom surface 45/topical gelatinous layer 60 of the device, enter the skin wherein the pressure therein forces the liquid anesthetic to the gingiva.

Referring the FIG. 12, a base portion of the device 10 may be surrounded by an absorbent swab 70 (this may be placed in rectangular fashion as a protective barrier to prevent saliva intrusion) to enable absorption into the gingiva. The absorbent material 70 may include be tube-shaped and carefully wrapped along the perimeter of the topical anesthetic (to prevent saliva from diluting the mixture). This first mode numbs the gingiva so that when the device 10 is pressed down there is less pain because the topical has taken effect. This first mode can only take effect within the first 2mm-3 mm of gingiva which is why when the micro needles 20 are deployed it is less painful. When said device is compressed (as shown), the compression forces the analgesic through the aforementioned micro needles 20 thereon along with the topical anesthetic painlessly. During a second mode the user positions the device onto the gingiva inside the absorbent material and on top of the topical anesthetic, whereby the user compresses the pressable reservoir for pushing the micro needles and analgesic into the gingiva. Thereafter, the user releases the pressable reservoir 10 and retracts the micro needles 20.

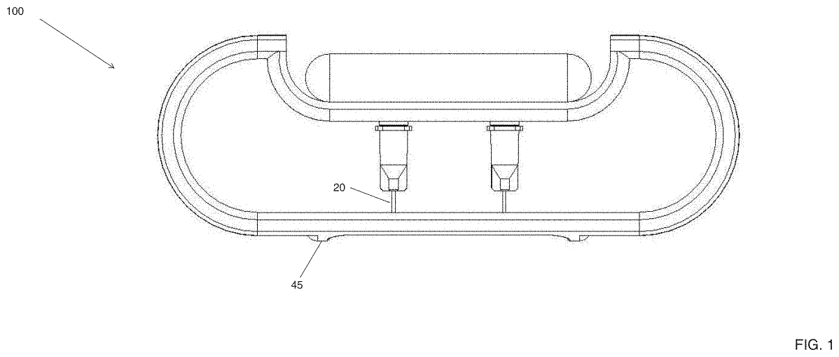

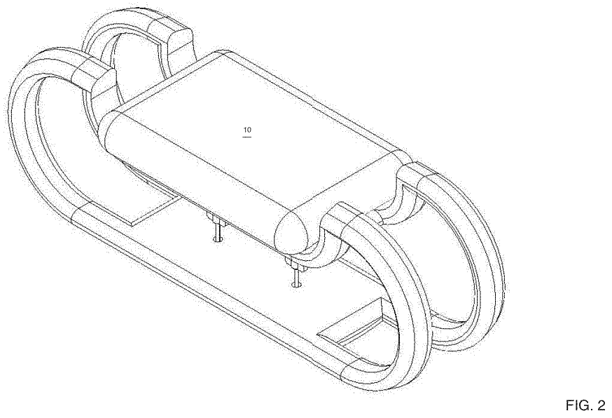

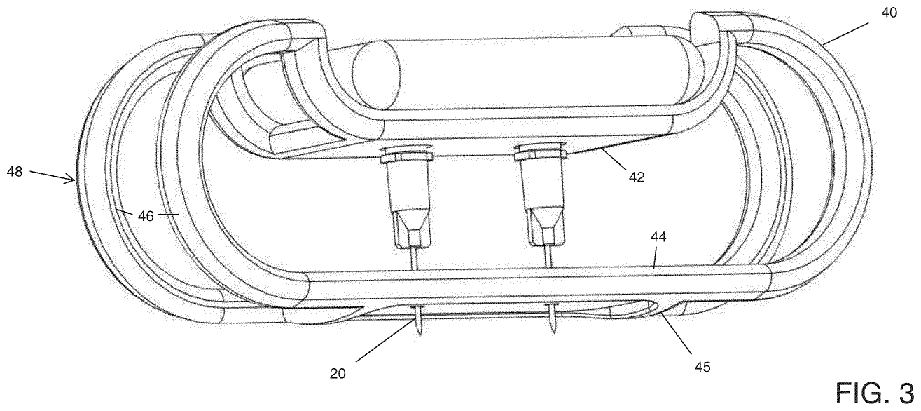

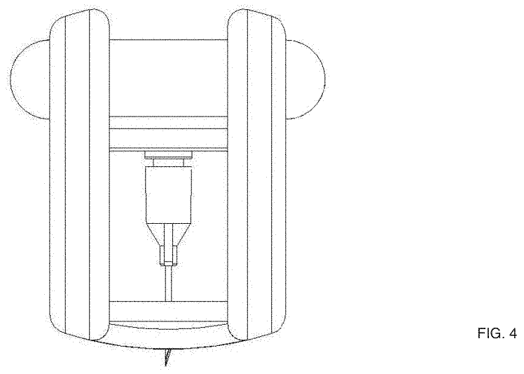

In this description, the directional prepositions of up, upwardly, down, downwardly, front, back, top, upper, bottom, lower, left, right and other such terms refer to the device as it is oriented and appears in the drawings, specifically FIG. 1. In FIG. 1, the directional preposition “upward” is toward the top margin of FIG. 1, while the directional prepositions of “downward” (or bottom) is toward the bottom of FIG. 1. In any event, the directional prepositions are used for convenience only; they are not intended to be limiting or to imply that the device has to be used or positioned in any particular orientation. Conventional components of the disclosure are elements that are well-known in the prior art and will not be discussed in detail for this disclosure.

In certain embodiments, a vibrating feature to attempt to use the gate control theory of pain.

As used in this application, the term “about” or “approximately” refers to a range of values within plus or minus 10% of the specified number. And the term “substantially” refers to up to 90% or more of an entirety.

It is additionally noted and anticipated that although the device is shown in its most simple form, various components and aspects of the device may be differently shaped or slightly modified, such as the different shapes of the bottom surface 45 shown or the shape of the frames 40 shown. As such those skilled in the art will appreciate the descriptions and depictions set forth in this disclosure or merely meant to portray examples of preferred modes within the overall scope and intent of the system and method for analgesic delivery into gingiva, and are not to be considered limiting in any manner. While all of the fundamental characteristics and features have been shown and described herein, with reference to particular embodiments thereof, a latitude of modification, various changes and substitutions are intended in the foregoing disclosure and it will be apparent that in some instances, some features of the system and method for analgesic delivery into gingiva may be employed without a corresponding use of other features without departing from the scope as set forth. It should also be understood that various substitutions, modifications, and variations may be made by those skilled in the art without departing from the scope of the disclosure.

It should be understood, of course, that the foregoing relates to exemplary embodiments of the invention and that modifications may be made without departing from the spirit and scope of the invention as set forth in the following claims.

What is claimed is:

1. A device, comprising:
a pressable reservoir having a plurality of outlets, wherein the pressable reservoir is movable between an undeformed condition and a deformed condition; and
a frame having a support portion spaced apart from a base portion, wherein an upper surface of the support portion is connected to a bottom surface of the pressable reservoir, wherein moving from the undeformed condition to the deformed condition reduces a volume of the pressable reservoir and moves the support portion toward the base portion.

2. The device of claim 1, further comprising a plurality of needles fluidly coupled to the plurality of outlets, wherein the termini of the plurality of needles is adjacent to bottom surface.

3. The device of claim 2, further comprising a layer of topical gelatinous anesthesia disposed along the base portion in such a way that said layer terminates at a contact surface approximately flush with the bottom surface.

4. The device of claim 3, wherein said termini move from upward of the contact surface to downward thereof upon moving between the undeformed condition to the deformed condition.

5. The device of claim 3, further comprising a cavity along the base portion, wherein the cavity houses said layer.

6. The device of claim 5, wherein the frame has curved ends interconnecting the base portion and the support portion.

7. The device of claim 6, wherein the frame comprises two laterally spaced apart rails.

8. The device of claim 7, further comprising an absorptive material adjacent said layer.

9. The device of claim 8, further comprising a liquid anesthetic housed in the pressable reservoir.

10. A method for delivering analgesic into a gingiva, the method comprising:
contacting said layer of the device of claim 9 against a surficial surface of the gingiva for between two to six minutes; and
thereafter moving the pressable reservoir to the deformed condition.
